(19) United States
(12) Patent Application Publication
(10) Pub. No.: US 2014/0296939 A1
(43) Pub. Date: Oct. 2, 2014

(54) NERVE STIMULATION SYSTEM AND WIREBOARD THEREOF

(71) Applicant: Taiwan Advanced Sterilization Technology, Inc., Taichung City (TW)

(72) Inventors: Peng-Chieh Wu, Taipei City (TW); Wen-Hsuan Liao, Taichung City (TW); Wei-Hsuan Liao, Taichung City (TW); Yung-Ting Kuo, Taipei City (TW)

(73) Assignee: Taiwan Advanced Sterilization Technology, Inc., Taichung City (TW)

(21) Appl. No.: 14/217,584

(22) Filed: Mar. 18, 2014

(30) Foreign Application Priority Data
Mar. 28, 2013 (TW) 102111150

(51) Int. Cl. A61N 1/36

(52) U.S. Cl. CPC A61N 1/36125; A61N 1/36139
USPC 607/60

(57) ABSTRACT

A nerve stimulation system for a biological subject includes a stimulator to be disposed in the biological subject, sensing units to be disposed on the biological subject for obtaining biological information thereof, and an evaluation device. The evaluation device adjusts a parameter according to the biological information received from the sensing units, and generates a control signal indicating the parameter adjusted thereby. The stimulator wirelessly receives the control signal from the evaluation device, and generates a stimulation signal to stimulate the biological subject according to the parameter adjusted by the evaluation device.

### CROSS-REFERENCE TO RELATED APPLICATION

This application claims priority to Taiwanese Application No. 102111150, filed on Mar. 28, 2013.

### BACKGROUND OF THE INVENTION

1. Field of the Invention

The invention relates to a nerve stimulation system for a biological subject.

2. Description of the Related Art

Brain treatments may be classified into invasive treatments (e.g., vagus nerve stimulation (VNS), deep brain stimulation (DBS) and implantable biochips), and non-invasive treatments (e.g., transcranial magnetic stimulation (TMS)).

In the case of VNS, a stimulator is disposed at a vagus nerve site of the neck, and parameters of the stimulator are controlled through wireless communication to stimulate the vagus nerves in various levels for observation of the brain wave.

Clinically, after a patient receives the VNS treatment, doctors will evaluate possible variation of the patient's brain wave upon the patient's reaction, personal experiences and instinct as a reference for adjustment of the parameters of the stimulator. However, it is difficult to obtain precise evaluation only based on the personal experiences and instinct, and better brain wave evaluation may be obtained by analyzing energy variation of the brain wave on both the spectrum and the time-domain. Without a precise brain wave analysis, doctors may make an incorrect judgment and thus determine an improper set of parameters, resulting in waste of medical resources and delay of treatment progress of the patient.

### SUMMARY OF THE INVENTION

Therefore, an object of the present invention is to provide a nerve stimulation system that may effectively adjust parameters of a stimulator for stimulation of a biological subject.

According to one aspect of the present invention, a nerve stimulation system is adapted for a biological subject, and comprises:

a stimulator to be disposed in a body of the biological subject;

a plurality of sensing units each to be disposed on the body of the biological subject for obtaining biological information thereof, the sensing units including a first sensing unit to be disposed on ahead portion of the biological subject for obtaining first biological information, and a second sensing unit to be disposed adjacent to the stimulator for obtaining second biological information; and

an evaluation device including:
a signal processor configured to receive the biological information from the sensing units, and to adjust the first biological information according to the second biological information, so as to obtain an analysis signal;
a signal generator coupled to the signal processor and configured to adjust a parameter based upon the analysis signal, and to generate a control signal indicating the parameter adjusted thereby; and
a transceiver coupled to the signal generator and configured to wirelessly transmit the control signal to the stimulator.

The stimulator is configured to generate a stimulation signal to stimulate the biological subject according to the parameter adjusted by the signal generator and indicated by the control signal.

Another object of the present invention is to provide a wire connection board that is adapted for use in the nerve stimulation system of the present invention.

According to another aspect of the present invention, a wire connection board is adapted for use with an evaluation device that is used to evaluate a biological subject having a stimulator disposed in a body of the biological subject. The wire connection board comprises:

a first mark to represent a head portion of the biological subject;

a second mark to represent the stimulator;

a first connection hole adjacent to the first mark and to be coupled to a wire used to transmit biological information from the head portion of the biological subject; and

a second connection hole adjacent to the second mark and to be coupled to a wire used to transmit biological information from a portion of the body of the biological subject adjacent to the stimulator.

The wire connection board transmits the biological information received thereby to the evaluation device for evaluation.

Yet another object of the present invention is to provide a nerve stimulation system for use with a stimulator disposed in a body of a biological subject, and with a plurality of sensing units that obtain biological information of the biological subject. The biological information includes first and second biological information respectively associated with a head portion of the biological subject and a portion of the body of the biological subject that is adjacent to the stimulator. The nerve stimulation system comprises:

a signal processor configured to receive the biological information from the sensing units, and to adjust the first biological information according to the second biological information, so as to obtain an analysis signal; and

a signal generator coupled to the signal processor, and configured to adjust a parameter based upon the analysis signal, the parameter thus adjusted to serve as a basis for stimulation of the biological subject.

### BRIEF DESCRIPTION OF THE DRAWINGS

Other features and advantages of the present invention will become apparent in the following detailed description of the preferred embodiments with reference to the accompanying drawings, of which:

FIG. 1 is a block diagram illustrating a first preferred embodiment of the nerve stimulation system according to the present invention;

FIG. 2 is a schematic diagram illustrating that a stimulator is disposed at a vagus nerve site;

FIG. 3 is a schematic diagram illustrating that sensing units are disposed at a head portion of a biological subject, a body portion adjacent to a heart of the biological subject, and another body portion adjacent to the stimulator;

FIG. 4 is a schematic diagram showing distribution of connection holes of a wire connection board of the present invention;

FIG. 5 is a block diagram illustrating another implementation of the first preferred embodiment; and

FIG. 6 is a block diagram illustrating a second preferred embodiment of the nerve stimulation system according to the present invention.

### DETAILED DESCRIPTION OF THE PREFERRED EMBODIMENTS

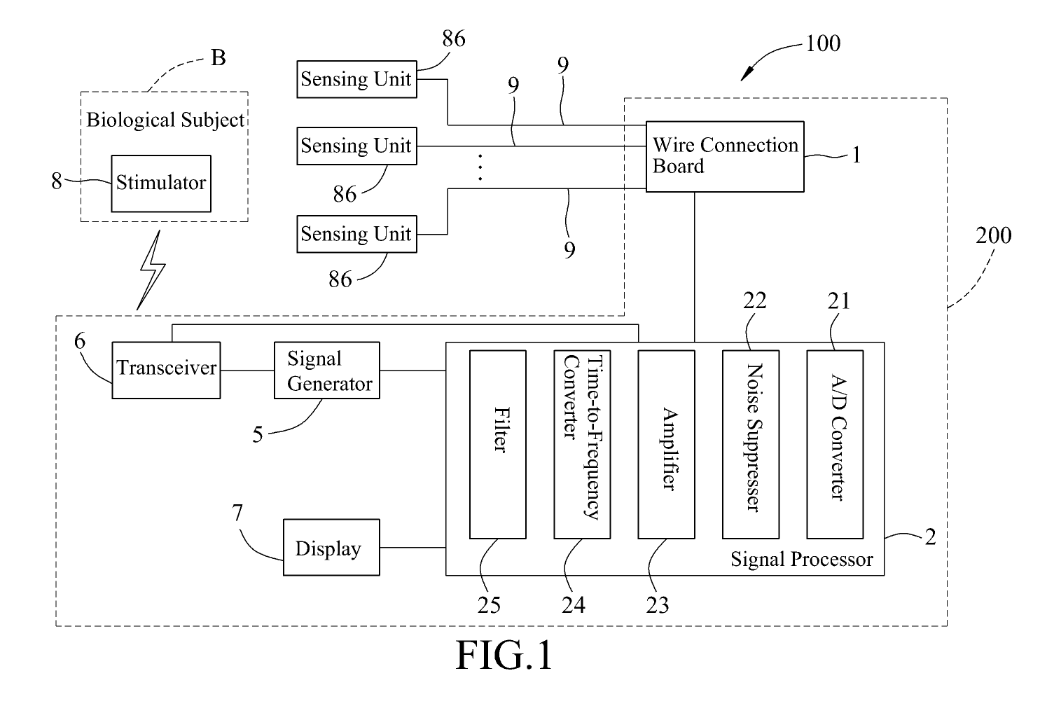

Referring to FIG. 1, the first preferred embodiment of the nerve stimulation system 100 according to this invention is shown to be adapted for a biological subject B, and includes an evaluation device 200, a stimulator 8 to be disposed in a body of the biological subject B, a plurality of sensing units 86 to be disposed on the body of the biological subject B, and a plurality of wires 9 corresponding respectively to the sensing units 86.

The evaluation device 200 includes a wire connection board 1, a signal processor 2, a signal generator 5, a transceiver 6 and a display 7. The signal generator 5 is coupled to the signal processor 2. The transceiver 6 is coupled to the signal generator 5 and the signal processor 2, and is configured to wirelessly communicate with the stimulator 8. The display 7 is coupled to the signal processor 2 for displaying processing results of the signal processor 2. The signal processor 2 includes an analog-to-digital converter (A/D converter) 21, a noise suppressor 22, an amplifier 23, a time-to-frequency converter 24 and a filter 25.

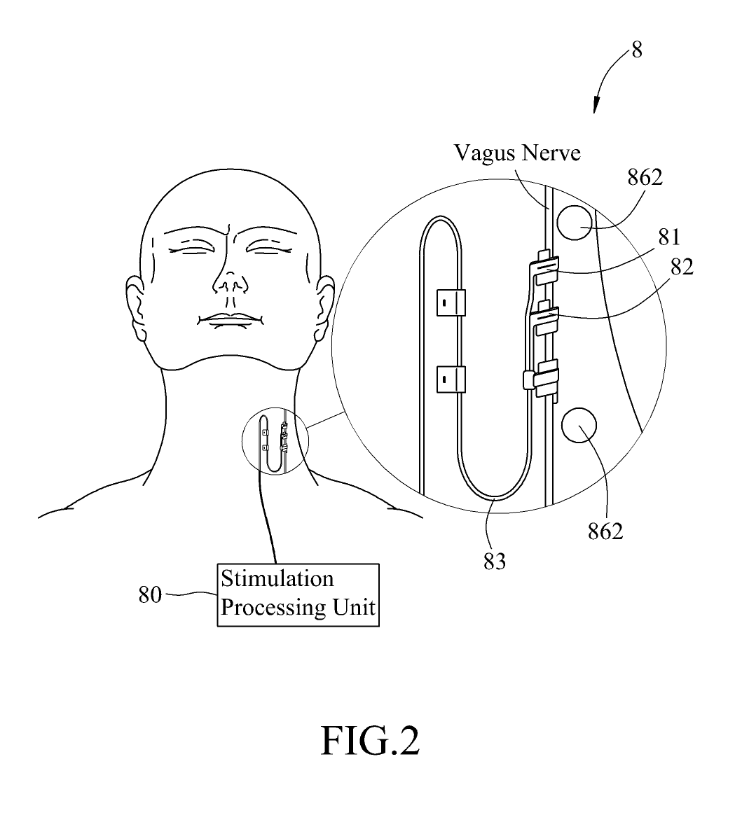

Referring to FIG. 2, preferably, the biological subject B is a human subject, but is not limited thereto. The stimulator 8 includes a stimulation processing unit 80, a stimulation wire 83, and first and second stimulating components 81, 82 spaced apart from each other and disposed on the vagus nerve of a neck portion of the biological subject B. The first and second stimulating components 81, 82 are connected to the stimulation processing unit 80 via the stimulation wire 83. In other embodiments, the first and second stimulating components 81, 82 may be disposed on the nerve sites of other portions of the biological subject B.

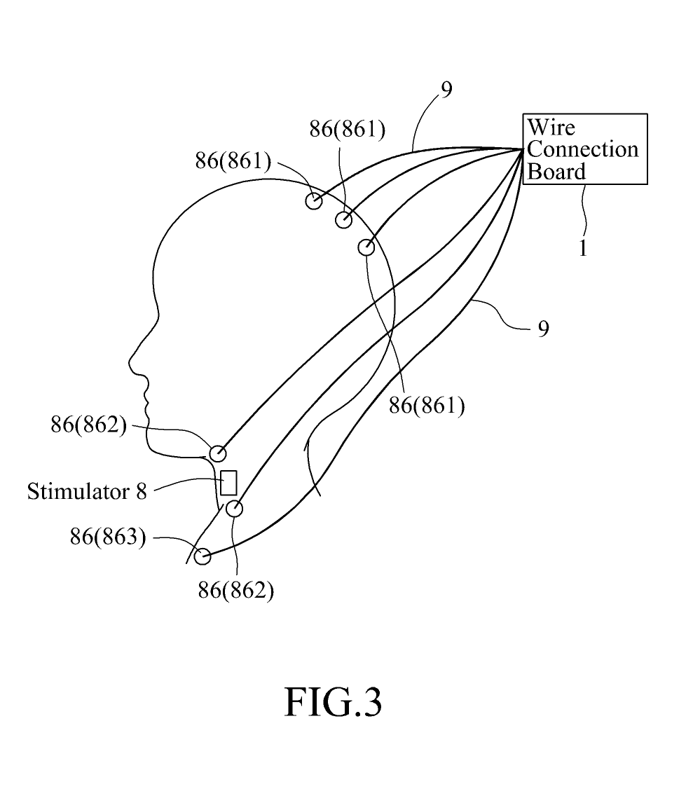

Further referring to FIG. 3, each of the sensing unit 86 is disposed on the body of the biological subject B for obtaining biological information thereof, and each of the wires 9 has two ends coupled respectively to the corresponding sensing unit 86 and the wire connection board 1 for transmission of the biological information from the corresponding sensing unit 86 to the signal processor 2 through the wire connection board 1. In this embodiment, the sensing units 86 include first sensing units 861 disposed on a head portion of the biological subject B for obtaining first biological information, a pair of second sensing units 862 disposed respectively adjacent to the first and second stimulating components 81, 82 of the stimulator 8 for obtaining second biological information, and a third sensing unit 863 disposed adjacent to a heart of the biological subject B for obtaining third biological information. However, numbers of the first, second, and third sensing units are not limited to those of the preferred embodiment, and may vary according to the actual requirement. For example, there maybe a hundred of the first sensing units 861, only one or any number of the second sensing units 862, and only one or any number of the third sensing units 863. When two third sensing units 863 are in use, the third sensing units 863 usually are disposed respectively proximate to the left chest and right chest, or disposed to be spaced apart from each other on the heart portion of the biological subject B.

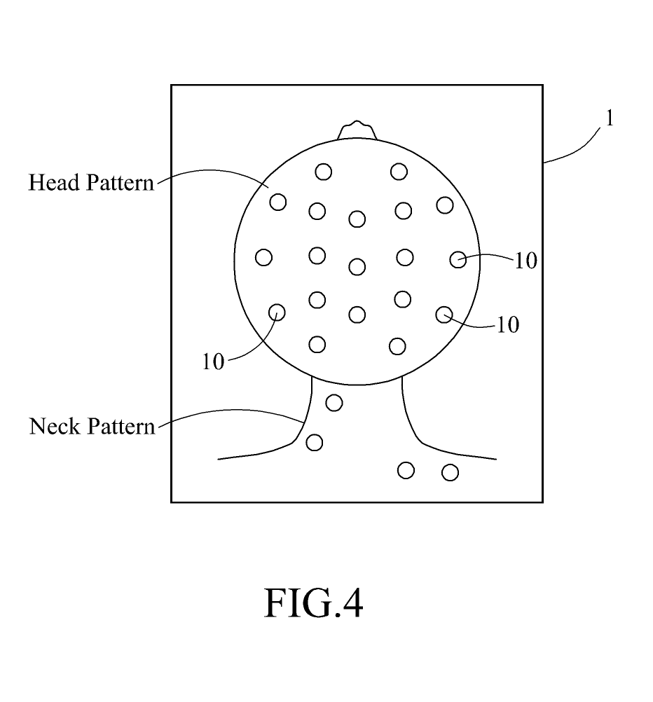

Referring to FIG. 4, the wire connection board 1 has a plurality of connection holes 10 corresponding respectively to the wires 9. To facilitate operation of users, the wire connection board 1 is preferably formed with a first mark representing the head portion of the biological subject B, and a second mark representing the stimulator 8. In this embodiment, the first mark is a head pattern and the second mark is a neck pattern. The connection holes 10 of the wire connection board 1 include first connection holes formed within the head pattern and coupled to a first portion of the wires 9 corresponding to the first sensing units 861, second connection holes formed within the neck pattern and coupled to a second portion of the wires 9 corresponding to the second sensing units 862, and third connection holes that are formed at a side of the neck pattern away from the head pattern, and that are coupled to a third portion of the wires 9 corresponding to the third sensing unit 863. The number of the connection holes 10 corresponds to the number of the wires 9, and is not limited to that shown in FIG. 4.

The nerve stimulation system 100 generally has the following three operation modes: external control mode, research mode and analysis mode.

In the external control mode, the signal generator 5 is externally controlled to adjust a parameter, and the transceiver 6 wirelessly transmits a control signal indicating the parameter to the stimulator 8. After receiving the control signal, the stimulation processing unit 80 will generate a confirmation signal for the signal processor 2 in response, and will generate a stimulation signal corresponding to the parameter and according to the control signal for the stimulating components 81, 82 to stimulate the vagus nerve.

In the research mode, operation of the nerve stimulation system 100 is similar to that in the external control mode, but the stimulation processing unit 80 further provides to the signal processor 2 information associated with the frequency of the stimulation signal, corresponding parameters, start time of the stimulation, end time of the stimulation, as well as other characteristics of the stimulator 8, to serve as a basis for further research.

In the analysis mode, the detailed operation of the nerve stimulation system 100 is described as follows.

Referring to FIG. 1, the A/D converter 21 is configured to convert the first, second and third biological information that are received from the wire connection board 1 into first, second and third digital signals, respectively. The noise suppressor 22 is configured to obtain first, second and third noise suppressed signals according to the first, second and third digital signals, respectively. The amplifier 23 is configured to obtain first, second and third amplified signals according to the first, second and third noise suppressed signals, respectively. The time-to-frequency converter 24 is configured to obtain first, second and third frequency-domain signals according to the first, second and third amplified signals within an observation time period, respectively.

The filter 25 is configured to adjust the first frequency-domain signal according to the second and third frequency-domain signals, so as to obtain a filter signal serving as an analysis signal. In detail, the filter 25 filters out the second and third frequency-domain signals from the first frequency-domain signal, so as to obtain the filter signal serving as the analysis signal. Then, the signal generator 5 is configured to adjust a parameter based upon the analysis signal, and to generate a control signal indicating the parameter adjusted thereby. In this embodiment, the parameter is loaded into the control signal. The transceiver 6 is configured to receive the control signal from the signal generator 5 and wirelessly transmit the control signal to the stimulator 8.

The stimulation processing unit 80 of the stimulator is configured to wirelessly receive the control signal, to generate the confirmation signal for the signal processor 2 after receipt of the control signal, to obtain the parameter therefrom for generating the stimulation signal with corresponding characteristics accordingly, and to transmit the stimulation signal to the stimulating components 81, 82 through the stimulation wire 83 to stimulate the vagus nerve of the biological subject B. The characteristics of the stimulation signal may be a signal frequency, a signal wave form, a signal amplitude, etc . . . . After stimulating the biological subject B, the stimulation processing unit 80 further provides information associated with the frequency of the stimulation signal, stimulation parameters, start time of the stimulation, end time of the stimulation, as well as other characteristics of the stimulator 8, to the signal processor 2 through the transceiver 6. The signal processor 2 may then determine the observation time period according to the start time and the end time of the stimulation.

In this embodiment, the transceiver 6 has a transmission protocol matching with that of the stimulation processing unit 80, and the control signal has a format conforming with the protocol. The protocol may be bluetooth, ZigBee, radio frequency identification (RFID), infrared, etc . . . .

It should be noted that, since the brain wave will be interfered by the stimulation signal and the heartbeat, the filter 25 is used to filter out the second and third frequency-domain signals from the first frequency-domain signal for obtaining the analysis signal effectively reflecting the brain wave.

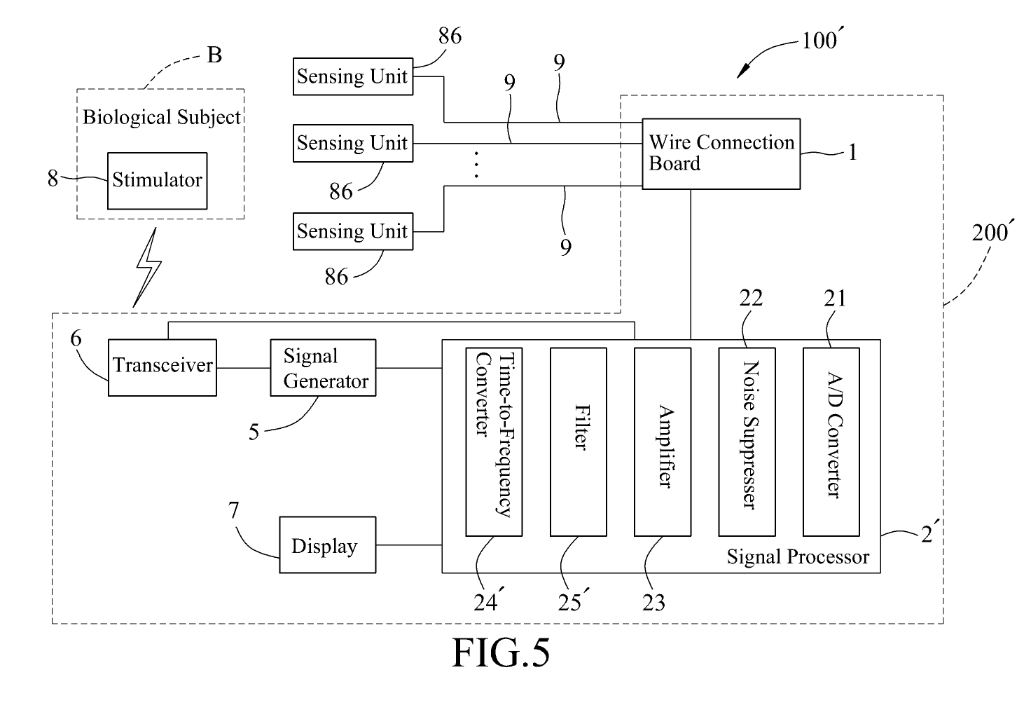

In this implementation, time-to-frequency conversion is performed prior to the filter operation, but in other implementations, as illustrated in FIG. 5, the filter 25′ may be used to filter out the second and third amplified signals from the first amplified signal in the time domain to obtain the filter signal, and then the time-to-frequency converter 24′ then converts the filter signal into a frequency-domain signal serving as the analysis signal.

It should be noted that the time-to-frequency converter 24, 24′ may employ fast Fourier transform (FFT), Hilbert Huang transform (HHT), or other methods to convert signals from the time domain to the frequency domain.

The signal processor 2, the signal generator 5 and the transceiver 6 may be integrated in an electronic device, such as a smartphone or a computer. As described above, the first mark is a head pattern and the second mark is a neck pattern in this embodiment to facilitate visual operation. In other applications, a plurality of marks may be used to represent the corresponding sensed portion of the biological subject B for facilitating connection of the wires 9 to the wire connection board 1. The sensed portion may be the head portion, the heart portion, the neck portion, the vicinity of the stimulator 8, or other portions of the biological subject B. Each of the marks maybe a pattern, a text, a symbol, a color or a shape. For example, a text representing the head portion may be disposed adjacent to one of the connection holes 10 to guide users to connect the wire 9 corresponding to the head portion to that connection hole 10. In another example, each of the connection holes 10 has the same color as the corresponding wire 9. In yet another example, each of the connection holes 10 is formed with a shape matching with a shape of the connector of the corresponding wire 9 for facilitating plugging operation.

Although the stimulator 8 is disposed at the vagus nerve site of the neck portion in this embodiment, it may be disposed at the vagus nerve site of another portion of the biological subject B, or other types of the nerves in other applications.

Although the evaluation device 200 uses a plurality of wires 9 and corresponding sensing units 86 for connection to the biological subject B in this embodiment, it may use only one cable for electrical connection to a sensing helmet having a plurality of sensing units 86 (not shown). When the biological subject B wears the sensing helmet, the sensing units 86 provide the sensed biological information to the evaluation device 200 through the cable.

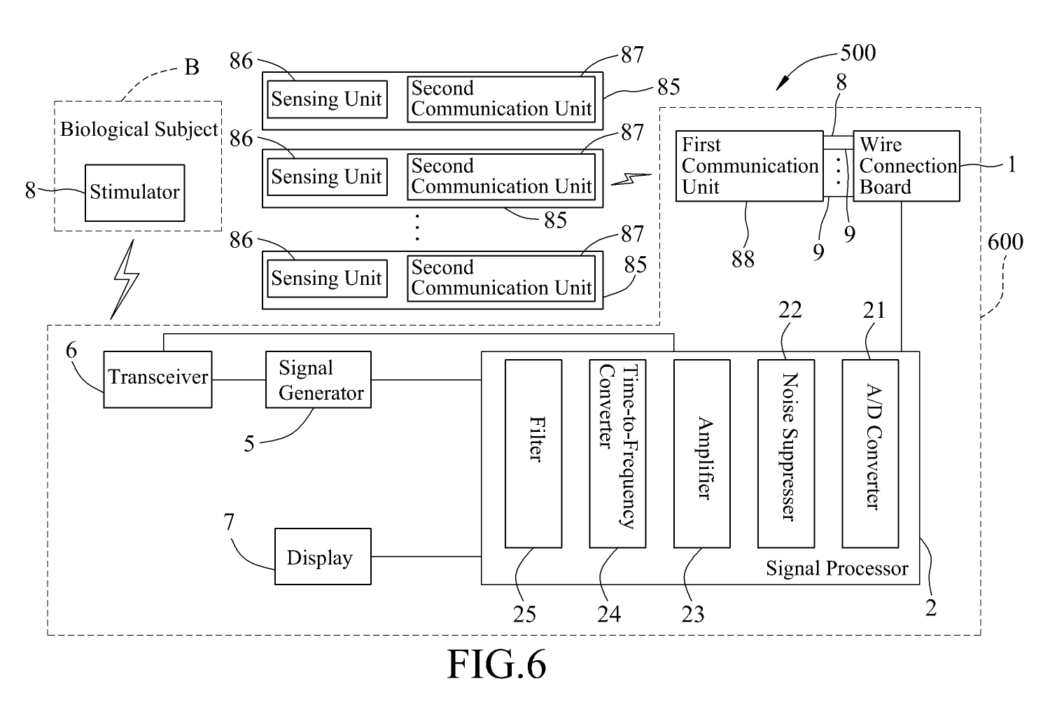

Referring to FIG. 6, a second preferred embodiment of the nerve stimulation system 500 according to the present invention is shown to differ from the first preferred embodiment in that: the evaluation device 600 further includes a first communication unit 88, and the wires 9 are coupled between the first communication unit 88 and the wire connection board 1. The nerve stimulation system 500 includes a plurality of detectors 85 for detection of the biological subject B, and each of the detectors 85 includes a sensing unit 86 and a second communication unit 87. Each of the sensing units 86 is configured to obtain biological information from the biological subject B, and the corresponding second communication unit 87 is configured to wirelessly transmit the biological information thus obtained to the first communication unit 88 of the evaluation device 600. Then, the first communication unit 88 transmits the biological information to the wire connection board 1 through the wires 9.

In other implementations, the wire connection board and the wires 9 may be omitted, and the first communication unit 88 may be configured to directly transmit the biological information to the signal processor 2. The wireless protocol used between the first and second communication units 88, 87 may be bluetooth, ZigBee, RFID, infrared, etc . . . . Furthermore, the first communication unit 88 and the transceiver 6 maybe integrated in a transceiving device (not shown) .

To sum up, the nerve stimulation system of this invention uses the signal processor 2 to obtain the analysis signal according to the biological information from the sensing units 86 disposed on the head portion, the heart portion, and the body portion adjacent to the stimulator 8, so that the signal generator 5 can effectively adjust the parameter to thus generate the stimulation signal to stimulate the vagus nerve of the biological subject B, thereby being helpful for brain treatment. In addition, the design of the wire connection board 1 facilitates operation of the nerve stimulation system, so as to reduce possibility of mistakes attributed to carelessness.

While the present invention has been described in connection with what are considered the most practical and preferred embodiments, it is understood that this invention is not limited to the disclosed embodiments but is intended to cover various arrangements included within the spirit and scope of the broadest interpretation so as to encompass all such modifications and equivalent arrangements.

What is claimed is:

1. A nerve stimulation system for a biological subject, said nerve stimulation system comprising:
a stimulator to be disposed in a body of the biological subject;
a plurality of sensing units each to be disposed on the body of the biological subject for obtaining biological information thereof, said sensing units including a first sensing unit to be disposed on ahead portion of the biological subject for obtaining first biological information, and a second sensing unit to be disposed adjacent to said stimulator for obtaining second biological information; and
an evaluation device including:
a signal processor configured to receive the biological information from said sensing units, and to adjust the first biological information according to the second biological information, so as to obtain an analysis signal;
a signal generator coupled to said signal processor and configured to adjust a parameter based upon the analysis signal, and to generate a control signal indicating the parameter adjusted thereby; and
a transceiver coupled to said signal generator and configured to wirelessly transmit the control signal to said stimulator;
wherein said stimulator is configured to generate a stimulation signal to stimulate the biological subject according to the parameter adjusted by said signal generator and indicated by the control signal.

2. The nerve stimulation system as claimed in claim 1, wherein said signal processor is configured to convert the first biological information into a first frequency-domain signal, to convert the second biological information into a second frequency-domain signal, and to filter out the second frequency-domain signal from the first frequency-domain signal.

3. The nerve stimulation system as claimed in claim 1, wherein said signal processor is configured to process the first biological information according to the second biological information for obtaining a filter signal, and to perform a time-to-frequency domain conversion on the filter signal, so as to obtain the analysis signal.

4. The nerve stimulation system as claimed in claim 1, wherein said sensing units further include a third sensing unit to be disposed adjacent to a heart of the biological subject for obtaining third biological information, and said signal processor is configured to adjust the first biological information according to the second and third biological information, so as to obtain the analysis signal.

5. The nerve stimulation system as claimed in claim 4, wherein said signal processor is configured to convert the first biological information into a first frequency-domain signal, to convert the second biological information into a second frequency-domain signal, to convert the third biological information into a third frequency-domain signal, and to filter out the second and third frequency-domain signals from the first frequency-domain signal, so as to obtain the analysis signal.

6. The nerve stimulation system as claimed in claim 4, wherein said signal processor is configured to perform a filter operation on the first biological information according to the second and third biological information for obtaining a filter signal, and to perform a time-to-frequency domain conversion on the filter signal, so as to obtain the analysis signal.

7. The nerve stimulation system as claimed in claim 1, further comprising a plurality of wires corresponding respectively to said sensing units, wherein said evaluation device further includes a wire connection board coupled to said signal processor;
each of said wires having two ends coupled respectively to the corresponding one of said sensing units and said wire connection board for transmission of the biological information from the corresponding one of said sensing units to said signal processor through said wire connection board.

8. The nerve stimulation system as claimed in claim 7, wherein said wire connection board has
a first mark representing the head portion of the biological subject,
a second mark representing said stimulator,
a first connection hole adjacent to said first mark and coupled to a first portion of said wires corresponding to said first sensing unit, and
a second connection hole adjacent to said second mark and coupled to a second portion of said wires corresponding to said second sensing unit.

9. The nerve stimulation system as claimed in claim 8, wherein said sensing units further include a third sensing unit to be disposed adjacent to a heart of the biological subject, said first mark being a head pattern, said wire connection board further having a neck pattern and a third connection hole; and
wherein said first connection hole is formed within said head pattern, said third connection hole being formed at a side of said neck pattern away from said head pattern, and being coupled to a third portion of said wires corresponding to said third sensing unit.

10. The nerve stimulation system as claimed in claim 7, wherein said stimulator is to be disposed at a nerve site of the biological subject to stimulate the nerve site of the biological subject, said wires including two wires coupled to said second sensing unit.

11. The nerve stimulation system as claimed in claim 1, wherein communication between said transceiver and said stimulator is performed using one of bluetooth, radio-frequency identification and infrared protocol.

12. The nerve stimulation system as claimed in claim 1, further comprising a plurality of first communication units corresponding respectively to said sensing units, wherein said evaluation device further includes a second communication unit coupled to said signal processor, each of said first communication units being configured to transmit wirelessly the biological information obtained by a corresponding one of said sensing units to said second communication unit.

13. A wire connection board for use with an evaluation device, the evaluation device being used to evaluate a biological subject having a stimulator disposed in a body of the biological subject, said wire connection board comprising:
a first mark to represent a head portion of the biological subject;
a second mark to represent the stimulator;
a first connection hole adjacent to said first mark and to be coupled to a wire used to transmit biological information from the head portion of the biological subject; and
a second connection hole adjacent to said second mark and to be coupled to a wire used to transmit biological information from a portion of the body of the biological subject adjacent to the stimulator;
wherein said wire connection board transmits the biological information received thereby to the evaluation device for evaluation.

14. The wire connection board as claimed in claim 13, wherein each of said first and second marks is one of a pattern, a text, a symbol, a color and a shape.

15. The wire connection board as claimed in claim 13, wherein said first mark is a head pattern, said wire connection board further having a neck pattern and a third connection hole;
wherein said first connection hole is formed within said head pattern, said third connection hole being formed at a side of said neck pattern away from said head pattern, and to be coupled to a wire used to transmit biological information corresponding to a heart of the biological subject.

16. A nerve stimulation system for use with a plurality of sensing units that obtain biological information of a biological subject, said nerve stimulation system comprising:
a stimulator to be disposed in a body of the biological subject; and
an evaluation device including:
a signal processor configured to receive the biological information from the sensing units, and to adjust first biological information of the biological information, which is associated with a head portion of the biological subject, according to second biological information of the biological information, which is associated with a portion of the body of the biological subject that is adjacent to said stimulator, so as to obtain an analysis signal;
a signal generator coupled to said signal processor and configured to adjust a parameter based upon the analysis signal, and to generate a control signal indicating the parameter adjusted thereby; and
a transceiver coupled to said signal generator for wireless transmission of the control signal to said stimulator;
wherein said stimulator is configured to generate a stimulation signal to stimulate the biological subject according to the parameter adjusted by said signal generator and indicated by the control signal.

17. The nerve stimulation system as claimed in claim 16, the biological information further including third biological information associated with a heart of the biological subject, wherein said signal processor is configured to convert the first biological information into a first frequency-domain signal, to convert the second biological information into a second frequency-domain signal, to convert the third biological information into a third frequency-domain signal, and to filter out the second and third frequency-domain signals from the first frequency-domain signal, so as to obtain the analysis signal.

18. The nerve stimulation system as claimed in claim 16, the biological information further including third biological information associated with a heart of the biological subject, wherein said signal processor is configured to perform filter operation on the first biological information according to the second and third biological information for obtaining a filter signal, and to perform a time-to-frequency domain conversion on the filter signal, so as to obtain the analysis signal.

19. A nerve stimulation system for use with a stimulator disposed in a body of a biological subject, and with a plurality of sensing units that obtain biological information of the biological subject, the biological information including first and second biological information respectively associated with a head portion of the biological subject and a portion of the body of the biological subject that is adjacent to the stimulator, said nerve stimulation system comprising:
a signal processor configured to receive the biological information from the sensing units, and to adjust the first biological information according to the second biological information, so as to obtain an analysis signal; and
a signal generator coupled to said signal processor, and configured to adjust a parameter based upon the analysis signal, the parameter thus adjusted to serve as a basis for stimulation of the biological subject.

20. The nerve stimulation system as claimed in claim 19, wherein said signal generator is further configured to generate a control signal indicating the parameter adjusted thereby, said nerve stimulation system further comprising a transceiver coupled to said signal generator for wireless transmission of the control signal to the stimulator.
